(19) United States
(12) Patent Application Publication
(10) Pub. No.: US 2025/0319088 A1
(43) Pub. Date: Oct. 16, 2025

(54) DAPRODUSTAT FOR REDUCING FATIGUE IN A SUBJECT WITH ANEMIA ASSOCIATED WITH CHRONIC KIDNEY DISEASE

(71) Applicant: GlaxoSmithKline Intellectual Property (No.2) Limited, Stevenage (GB)

(72) Inventors: Tony Tochi OKORO, Collegeville, PA (US); Alexander Ralph COBITZ, Collegeville, PA (US); Rubeen K. ISRANI, Collegeville, PA (US); Allison Barnes BLACKORBY, King of Prussia, PA (US); Vanja SIKIRICA, Collegeville, PA (US); Alistair Cameron LINDSAY, Collegeville, PA (US); Steven Ira BLUM, Collegeville, PA (US)

(21) Appl. No.: 18/571,386

(22) PCT Filed: Jun. 25, 2021
(86) PCT No.: PCT/IB2021/055708

§ 371 Date: Dec. 18, 2023

(51) Int. Cl. A61K 31/515; A61P 7/06; A61P 43/00

(52) U.S. Cl. CPC A61K 31/515; A61P 7/06; A61P 43/00

(57) ABSTRACT

The present invention relates to daprodustat or a pharmaceutically acceptable salt thereof for use in reducing fatigue in non-dialysis subjects with anemia associated with chronic kidney disease. In particular embodiments, the invention is directed to particular subject populations in which the subject has hsCRP≥6.60 mg/L at baseline and in which the subject has a haemoglobin concentration of ≤11 g/dL at baseline.

### FIELD OF THE INVENTION

The present invention relates to daprodustat or a pharmaceutically acceptable salt thereof for use in reducing fatigue in non-dialysis subjects with anemia associated with chronic kidney disease. In particular embodiments, the invention is directed to particular subject populations in which the subject has hsCRP≥6.60 mg/L at baseline and in which the subject has a haemoglobin concentration of ≤11 g/dL at baseline.

### BACKGROUND TO THE INVENTION

Instruments used to assess health related quality of life (HRQoL) include both patient-reported and clinician measured outcomes. The value of patient reported outcomes as opposed to clinician assessments is underscored by well-documented discrepancies in the assessment of problems and difficulties reported by patients and health care providers. As a result, several researchers in the field have emphasized the importance of patient assessments, suggesting that the patient himself is the expert when it comes to assessing his own quality of life. These researchers have stressed eliciting from the patient domains and concerns that may be unique to their condition, rather than relying on generic categories included in standardized HR-QOL instruments (for a review, see Finkelstein et al., 2009, Kidney International, 76(9):946-952). Despite this, at the present time, the 36-item short-form questionnaire (SF-36) is the most widely used HR-QoL instrument in chronic kidney disease (CKD) patients. It is not specific to any disease and includes 36 items that yield an 8-dimension profile on a 100-point scale, a higher score indicating a better perceived health state. The reliability, validity, and responsiveness of the SF-36 measure in patients with anemia of chronic kidney has been demonstrated (Finkelstein et al., Health Qual Life Outcomes, 2018; 16:111).

Using the SF-36, Pagels and colleagues report that all HRQoL dimensions deteriorated significantly across CKD stages, with the lowest scores in CKD stage 5. The largest reductions compared to matched controls were seen in ‘physical functioning,’ ‘role physical,’ ‘general health’ and in the physical component summary (PCS). The smallest disparities were seen in ‘mental health’ and ‘pain’. Patients with CKD stages 2-3 showed significantly decreased HRQoL compared to matched controls, with differences of large magnitude in ‘general health’ and PCS. Patients with CDK stage 4 demonstrated deteriorated scores with a large magnitude in ‘physical function,’ ‘general health’ and PCS compared to those with CKD stages 2-3. Pagels also reported that C-reactive protein (CRP)≥5 mg/L was among the most important predictors of impaired HRQoL. (Pagels et al. Health and Quality of Life Outcomes 2012, 10:71).

Anemia is a common complication of CKD. Common symptoms of anemia include low energy, fatigue, weakness, shortness of breath, dizziness, decreased exercise tolerance, impaired cognition, and decreased mental acuity. In addition, anemia has also been associated with diminished HR-QoL. Treatment of anemia with erythropoiesis-stimulating agents (ESAs) increases haemoglobin concentration, which may potentially relieve symptoms of anemia and improve the associated HR-QoL domains of Vitality and physical function. A number of studies have been conducted in which HR-QoL has been assessed in patients treated with ESAs using the SF-36 questionnaire. Early studies are reviewed by Gandra and colleagues (American Journal of Kidney Diseases, Vol 55, No 3 (March), 2010: pp 519-534). These studies vary in study design, with the majority being open label studies or studies comparing two management strategies, potentially introducing bias. Out of 11 studies reviewed, only 1 study reported clinically meaningful improvement in Vitality, 8 reported statistically significant improvements from baseline in Vitality, 1 study reported non-significant improvements from baseline in Vitality, and 1 study did not report results for energy or fatigue as study output. In view of the data, the FDA removed quality of life claims in labels of ESAs. This recommendation was based on the lack of controlled studies demonstrating an improvement in health-related quality of life measures.

The more recent CHOIR study did not include a placebo control and observed similar changes in the SF-36 Vitality Domain observed in the earlier studies (Singh et al., 2006, N. Engl. J. Med. 355 (20): 2085). Another recent study, in which the differences in haemoglobin concentration between treatment and control groups were smaller, showed smaller benefits that may not be clinically relevant (Drueke et al., 2006, N Engl J Med 355 (20): 2071). The TREAT study was a large, double blind study in which patients with type 2 diabetes mellitus, non-dialysis dependent chronic kidney disease and anemia were randomized to darbepoetin alfa or placebo. HRQoL was measured using multiple instruments including the SF-36. At 25 weeks, there was no significant difference in the mean change in SF-36 Vitality domain (5.3±20 versus 4.2±19 for darbepoetin alfa versus placebo, P=0.196) but the percentage of responders having a ≥5-point increase in SF-36 Vitality domain was 54% in patients receiving darbepoetin alfa compared to 49% of placebo patients (nominal P=0.027; Lewis et al., Clin J Am Soc Nephrol. 2011 April; 6(4): 845-855).

HIF-prolyl hydroxylase inhibitors are a newer class of agents being developed to treat anemia associated with CKD. Reports of the trials of the PHI inhibitor, vadadustat, have not included HRQoL outcomes. However, the placebo-controlled trials for the PHI inhibitor, roxadustat, did assess HRQoL outcomes. In a pooled analysis of the OLYMPUS, ANDES, and ALPS studies, patients treated with roxadustat experienced a small but statistically significant improvement in the SF-36 Vitality score (0-100 scoring) with a least squares mean difference of 0.96 points (95% CI 0.44 to 1.47) compared with those receiving placebo over 12 weeks. Whilst statistically significant, it is unlikely that this improvement is clinically relevant (Coyne et al., 2020, Health-Realted Quality of Life in Roxadustat-Treated Patients with Anemia and Non-Dialysis Dependent Chronic Kidney Disease, Oral presentation at the American Society of Nephrology Kidney Week, Oct. 22-25, 2020).

In summary, the reports relating changes in vitality paint a confusing picture, in which any improvements may depend upon the agent used and the study design including the degree of correction of anemia proposed.

Effective treatments of anemia of chronic kidney disease that also have a beneficial impact on vitality in non-dialysis patients are highly desirable.

### SUMMARY OF THE INVENTION

In a first aspect, the invention provides daprodustat or a pharmaceutically acceptable salt thereof for use in reducing fatigue in a subject with anemia associated with chronic kidney disease which subject is not on dialysis.

In particular embodiments, the invention is directed to a particular patient population in which the patient has hsCRP≥6.60 mg/L at baseline.

### DESCRIPTION OF DRAWINGS/FIGURES

FIG. 1 is a line plot of mean haemoglobin concentration (g/dL) by treatment and visit. Daprodustat data is represented by circles/dotted lines and placebo data is represented by triangles/solid lines.

FIG. 2 is a line plot of mean SF-36 vitality subscore values by treatment and visit. Daprodustat data is represented by circles/dotted lines and placebo data is represented by triangles/solid lines.

FIG. 3 is a Forest Plot of Adjusted Means from the hsCRP subgroup Analysis of Hemoglobin Change from Baseline to the Evaluation Period.

FIG. 4 is a Forest Plot of Adjusted Means from the Analysis of On-Treatment SF-36 Vitality Change from Baseline at Week 28 by hsCRP Subgroup.

FIG. 5 is a Forest Plot of Adjusted Means from the Analysis of On-Treatment SF-36 Vitality Change from Baseline at Week 28 by Haemoglobin subgroup.

### DETAILED DESCRIPTION OF THE INVENTION

Fatigue is a patient reported outcome and cannot be directly measured. Rather, it may be estimated by various instruments. The Vitality Domain of the short form SF-36 questionnaire (readily available from several sources, for example, https://www.rand.org/health-care/surveys_tools/mos/36-item-short-form/survey-instrument.html) provides a verified measure of fatigue. It provides a score from 0-100 in which a higher score denotes a decrease in fatigue. The Chronic Amemia Disease Anemia Questionnaire (CKD-AQ) is a patient reported outcome measure that has been developed to be specific to subjects with anemia of chronic kidney disease (Mathias et al. Journal of Patient-Reported Outcomes (2020) 4:64). The questions in the Tired/Low Energy/Weak Domain of the CKD-AQ are set out in Example 5. The Tired/Low Energy/Weak Domain of the CKD-AQ provides a measure of fatigue in which a higher score denotes a decrease in fatigue.

As explained in the section entitled “Background to the Invention”, treatment of anemia does not necessarily lead to a clinically meaningful improvement in vitality (reduction of fatigue). Instead, any improvement would appear to depend upon other factors, including the agent used to treat anemia and the degree of anemia correction. A post hoc analysis of the clinical study described in Example 1 demonstrates a very weak correlation between changes in haemoglobin concentration and improvement in Vitality (reduction of fatigue) as measured by either the SF-36 Vitality Domain Sub-score or the Tired/Low Energy/Weak Domain of the CKD-AQ (see Example 3). This confirms that treatment of anemia does not necessarily lead of a clinically meaningful improvement in vitality (reduction of fatigue). Despite this, it has been surprisingly found that daprodustat significantly reduces fatigue in non-dialysis subjects with anemia associated with chronic kidney disease (see Example 1).

Accordingly, in a first aspect, the invention provides daprodustat or a pharmaceutically acceptable salt thereof for use in reducing fatigue in a subject with anemia associated with chronic kidney disease which subject is not on dialysis. In one embodiment, fatigue is reduced at the end of the treatment period compared to baseline. In one embodiment, reduction of fatigue is observed when subjects are treated such that haemoglobin concentration in maintained in the range 10-12 g/dL. In a more particular embodiment, reductions in fatigue are observed when subjects are treated such that haemoglobin concentration is maintained in the range 11-12 g/dL.

In one embodiment, a reduction in fatigue refers to the situation where the subject's score on the vitality domain of the SF-36 questionnaire is increased at the end of the treatment period relative to baseline. In one particular embodiment, the treatment period is at least 12 weeks in duration.

Example 3 describes a search of the literature to identify thresholds for meaningful change (minimal clinically important difference or MCID) for the SF-36 Vitality Domain. This concludes (conservatively), that a within subject increase of six points in the SF-36 Vitality Domain is clinically relevant.

Accordingly, in one embodiment, a reduction in fatigue refers to the situation where the subject's score on the vitality domain of the SF-36 questionnaire is 6 points higher at the end of the treatment period compared to baseline. In another embodiment, a reduction in fatigue refers to the situation where the mean increase in the Vitality Domain of the SF-36 questionnaire is at least 6 points at the end of the treatment period relative to baseline in a population of subjects. In one embodiment, the population comprises at least 30 subjects. In the clinical study reported in Example 1, the adjusted mean change in the SF-36 Vitality domain from baseline (within treatment difference) was 7.29 (standard error=1.121) for subjects treated with daprodustat compared to 1.93 (standard error=1.161) for subjects randomized to placebo tablets.

In another embodiment, a reduction in fatigue refers to the situation where the mean score on the Vitality Domain of the SF-36 questionnaire is increased at the end of the treatment period relative to baseline in a population of subjects. In one embodiment, the population comprises at least 30 subjects. In one embodiment, the increase is at least a 6 point increase.

In an alternative embodiment, a reduction in fatigue is achieved where there is a higher responder rate for achieving at least a 6 point increase in the SF-36 Vitality Domain in subjects treated with daprodustat compared to placebo. In one embodiment, the difference in responder rate between daprodustat and placebo treated subjects is nominally significant with the one sided p-value being less than 0.025. In another embodiment, a reduction in fatigue is achieved where the percentage of subjects treated with daprodustat that achieve an increase of at least 6 points on the Vitality Domain of the SF-36 questionnaire is greater than 50%. In the clinical study Example 1, 58% of study participants treated with daprodustat demonstrated a ≥6.0 point difference from baseline to week 28 in the SF-36 Vitality Domain. The difference in response rate (dapro-placebo) was nominally significant (one sided p value=0.0049).

In another embodiment, a reduction in fatigue refers to the situation where the subject's score on the tired/low energy/weak domain of the CKD-anaemia questionnaire is increased at the end of the treatment period relative to baseline. In one particular embodiment, the treatment period is at least 12 weeks in duration. In one embodiment, a reduction in fatigue refers to the situation where the subject's score on the tired/low energy/weak domain of the CKD-AQ is increased by at least 5 points at the end of the treatment period relative to baseline. In another embodiment, the subject's score on the tired/low energy/weak domain of the CKD-AQ is increased by at least 8 points at the end of the treatment period relative to baseline. In another embodiment, the subject's score on the tired/low energy/weak domain of the CKD-AQ is increased by at least 11 points at the end of the treatment period relative to baseline.

In another embodiment, a reduction in fatigue refers to the situation where the mean increase tired/low energy/weak domain of the CKDAQ is at least 5 points at the end of the treatment period relative to baseline in a population of subjects. In a more particular embodiment, a reduction in fatigue refers to the situation where the mean increase tired/low energy/weak domain of the CKDAQ is at least 8 points at the end of the treatment period relative to baseline in a population of subjects. In a more particular embodiment, a reduction in fatigue refers to the situation where the mean increase tired/low energy/weak domain of the CKDAQ is at least 11 points at the end of the treatment period relative to baseline in a population of subjects. In particular embodiments, the population comprises at least 30 subjects. In the clinical study reported in Example 1, the adjusted mean change in the CKD-AQ Tired/Low Energy/Weak domain over the treatment period was 8.72 (standard error=1.086) for subjects treated with daprodustat compared to 2.81 (standard error 1.132) for subjects randomized to placebo tablets.

In yet another embodiment, a reduction in fatigue refers to the situation where the mean score in the tired/low energy/weak domain of the CKD-AQ is increased at the end of the treatment period relative to baseline in a population of subjects. In one embodiment, the population comprises at least 30 subjects. In one embodiment, the increase is at least a 5 point increase. In a more particular embodiment, the increase is at least a 8 point increase. In one embodiment, the increase is at least an 11 point increase.

Although a post hoc analysis (Example 3) demonstrated that there was a very weak correlation between changes in haemoglobin concentration and improvement in Vitality (reduction of fatigue) as measured by either the SF-36 Vitality Domain Sub-score or the Tired/Low Energy/Weak Domain of the CKD-AQ, the reductions in fatigue reported in Example 1 accompanied correction of anemia. This shows that a dosing schedule used for treatment of anemia is also appropriate for reducing fatigue, and will result in reductions that are clinically relevant and of the magnitude described above. In one embodiment, reductions in fatigue are observed when subjects are treated such that haemoglobin concentration in maintained in the range 10-12 g/dL. In a more particular embodiment, reductions in fatigue are observed when subjects are treated such that haemoglobin concentration is maintained in the range 11-12 g/dL.

In view of the weak correlation between changes in haemoglobin concentration and improvement in vitality (reduction in fatigue), it is believed that certain subject populations having relatively modest haemoglobin changes experience large reductions in fatigue.

Sub-group analyses of haemoglobin change and SF-36 Vitality Domain subscore, identified a population of subjects having hsCRP≥6.60 mg/L at baseline that responded to daprodustat with a relatively modest mean haemoglobin increase (the adjusted mean treatment difference in haemoglobin concentration was 1.25 g/dL (confidence intervals 0.92, 1.58) for subjects having hsCRP≥6.60 mg/L), but exhibited a comparably large increase in the SF-36 vitality subscore (the adjusted mean treatment difference in the SF-36 Vitality Domain subscore over the treatment period was 7.71 (confidence intervals 1.62, 13.80) for subjects having hsCRP≥6.60 mg/L). Accordingly, in one embodiment, the invention provides daprodustat or a pharmaceutically acceptable salt thereof for use in reducing fatigue in a subject with anemia associated with chronic kidney disease, wherein the subject has hsCRP≥6.60 mg/L at baseline. Table 8 shows that there were large reductions in hsCRP levels in this group raising the possibility that increases in vitality may be linked with hsCRP reduction. However, as explained in Example 1, no firm conclusions can be drawn because a) similar reductions in hsCRP levels were also observed in the placebo group with high baseline hsCRP (Table 8), and b) the reduction observed may (at least in part) be due to the subjects having the highest CRP levels not being evaluated at week 28/end of treatment, possibly because these subjects were rescued.

In another embodiment, the invention provides daprodustat or a pharmaceutically acceptable salt thereof for use in reducing inflammation in a subject with anemia associated with chronic kidney disease.

In the context of this invention, a reduction in inflammation reflects the situation where the hsCRP levels in a subject are reduced at the end of the treatment period compared to baseline. In a more particular embodiment, a reduction in inflammation reflects the situation where mean hsCRP levels in a subject are reduced at the end of the treatment period compared to baseline. In particular embodiments, the reduction relative to baseline is 20%, 30% or 40%. In a particular embodiment, the subject has hsCRP≥6.60 mg/L at baseline. In one embodiment, a reduction in inflammation is accompanied by a reduction in fatigue, as described herein.

Example 1 also discusses the impact of haemoglobin concentration upon adjusted mean treatment change in SF-36 Vitality Domain Sub-score. Whilst study design and the small number of subjects having haemoglobin>11 g/dL at baseline hampers firm conclusions being drawn, it seems likely that increases in SF36 Vitality Domain scores are most pronounced in subjects having baseline haemoglobin concentration of to ≤11 g/dL. Accordingly, in one embodiment, the invention provides daprodustat or a pharmaceutically acceptable salt thereof for use in reducing fatigue in a subject with anemia associated with chronic kidney disease, wherein the subject has a haemoglobin concentration of ≤11 g/dL at baseline. In a more particular embodiment, the subject has a haemoglobin concentration of ≤10 g/dL at baseline or of ≤9 g/dL at baseline. It is noted here that subjects having baseline haemoglobin<9 g/dL experienced an adjusted mean treatment difference in SF-36 Vitality domain subscore of 7.44 (confidence intervals −1.86, 16.73), subjects having baseline haemoglobin in the range≥9 to <10 g/dL experienced an adjusted mean treatment difference in SF-36 Vitality domain subscore of 5.25 (confidence intervals 0.85, 9.65), and subjects having baseline haemoglobin in the range≥10 to ≤11 g/dL experienced an adjusted mean treatment difference in SF-36 Vitality domain subscore of 6.01 (confidence intervals 0.83, 11.20). In particular embodiments, the invention provides daprodustat or a pharmaceutically acceptable salt thereof for use in reducing fatigue in a subject with anemia associated with chronic kidney disease, wherein the subject has a haemoglobin concentration in the range≥10 to ≤11 g/dL at baseline.

The study in Example 1 recruited subjects having CKD stages 3, 4 and 5. In one embodiment, the subject has CKD stage 3 or 4. In one embodiment, the subject has an GFR (or eGFR) of <60 ml/min/1.73 m². In a more particular embodiment, the subject has an GFR (or eGFR) of <45 ml/min/1.73 m². Accordingly, in one embodiment, the invention provides daprodustat or a pharmaceutically acceptable salt thereof for use in reducing fatigue in a subject with anemia associated with chronic kidney disease, wherein the subject is in CKD stage 3, 4 or 5. In particular embodiments, the subject is in CKD stage 3 or 4. In another embodiment, the invention provides daprodustat or a pharmaceutically acceptable salt thereof for use in reducing fatigue in a subject with anemia associated with chronic kidney disease, wherein the subject has a GFR (or eGFR) of <60 ml/min/1.73 m². In a more particular embodiment, the subject has an GFR (or eGFR) of <45 ml/min/1.73 m².

The invention also envisages narrower patient populations. For example, the invention provides daprodustat or a pharmaceutically acceptable salt thereof for use in reducing fatigue in a subject with anemia associated with chronic kidney disease, wherein the subject has one or more of:
a) hsCRP≥6.60 mg/L at baseline;
b) a haemoglobin concentration of ≤11 g/dL at baseline (e.g. <9 g/dL, ≥9 to <10 g/dL, <10 g/dL or ≥10 to ≤11 g/dL),
c) a GFR (or eGFR) of <45 ml/min/1.73 m² and
d) CKD stage 3, 4 or 5 (e.g. CKD stage 3 or 4)

In particular embodiments, the subject has:
a) and b);
a), b) and c);
a), b) and d);
b) and c); or
b) and d).

### Daprodustat

Daprodustat is the USAN, INN and JAN name for the compound N-((1,3-dicyclohexylhexahydro-2,4,6-trioxopyrimidin-5-yl)carbonyl)glycine (the IUPAC name for this compound is N-[(1,3-Dicyclohexylhexahydro-2,4,6-trioxopyrimidin-5-yl)carbonyl]glycine). Daprodustat exhibits keto/enol tautomerism and can also be named N-[(1,3-dicyclohexyl-6-hydroxy-2,4-dioxo-1,2,3,4-tetrahydro-5-pyrimidinyl)carbonyl]glycine. Where claims refer to N-[(1,3-dicyclohexyl-6-hydroxy-2,4-dioxo-1,2,3,4-tetrahydro-5-pyrimidinyl)carbonyl]glycine, all tautomers of N-[(1,3-dicyclohexyl-6-hydroxy-2,4-dioxo-1,2,3,4-tetrahydro-5-pyrimidinyl)carbonyl]glycine, including mixtures thereof, are intended to be encompassed within the scope of the invention.

In one embodiment, daprodustat or a pharmaceutically acceptable salt thereof are used in the methods of the invention. In a one embodiment, a pharmaceutically acceptable salt of daprodustat is used. In another embodiment, daprodustat free acid is used.

In one embodiment, daprodustat free acid is in crystalline form.

In a particular embodiment, the daprodustat free acid is a non-solvated crystalline form referred to as CS1. Form CS1 has an X-ray powder diffraction pattern that has characteristic peaks at 2theta values of 6.4°±0.2°, 7.5°±0.2°, and 7.9°±0.2° using CuKα radiation. In a more particular embodiment, the X-ray powder diffraction pattern of form CS1 has one or more additional characteristic peaks at 2theta values of 17.2°±0.2°, 21.0°±0.2°, 24.0°±0.2°, and 19.3°±0.2° using CuKα radiation. Form CS1 has an endothermic peak at around 242° C. as measured by differential scanning calorimetry using a heating rate of 10° C. min and a purge gas of nitrogen.

In another embodiment, the daprodustat free acid is a non-solvated crystalline form referred to as CS9. Form CS9 has an X-ray powder diffraction pattern that has characteristic peaks at 2theta values of 4.6°±0.2°, 6.6°±0.2°, and 21.1°±0.2° using CuKα radiation. In a more particular embodiment, the X-ray powder diffraction pattern for form CS9 has one or more additional characteristic peaks at 2theta values of 9.4°±0.2°, 20.2°±0.2°, and 24.2°±0.2° using CuKα radiation.

Forms CS1 and CS9 may be prepared from the free acid according to processes described in WO2019052133.

In another embodiment, the daprodustat free acid is a crystalline form referred to as Form 3. Form 3 has an X-ray powder diffraction pattern having peaks at 2-theta values of 4.5°±0.2°, 5.6°±0.2°, 9.0°±0.2° and 16.8°±0.2° using CuKα radiation. In a more particular embodiment, the X-ray powder diffraction pattern of Form 3 has one or more additional characteristic peaks at 2-theta values selected from 8.5°±0.2°, 11.2°±0.2°, 20.6°±0.2° and 24.7°±0.2° using CuKα radiation and/or a DSC endothermic peak with T onset at about 245.3° C.

In another embodiment, the daprodustat free acid is a crystalline form referred to as Form 4. Form 4 has an X-ray powder diffraction pattern having peaks at 2-theta values of 7.2°±0.2°, 11.5°±0.2°, 21.7°±0.2°, 22.9°±0.2°, 23.3°±0.2° and 25.8°±0.2° using CuKα radiation. In a more particular embodiment, the X-ray powder diffraction pattern of Form 4 has one or more additional characteristic peaks at 2-theta values selected from 6.3°±0.2°, 12.9°±0.2°, 16.5°±0.2°, 18.1°±0.2° and 19.7°±0.2° using CuKα radiation, and/or a DSC endothermic peak with T onset at about 243.9° C.

Forms 3 and 4 may be prepared as described in WO2020102302.

In another embodiment, the daprodustat free acid is a crystalline form referred to as form M. Form M has an X-ray powder diffraction pattern that has characteristic peaks at 2theta values of 4.7°±0.2°, 6.5°±0.2°, and 6.8°±0.2° using CuKα radiation. Form M may be prepared as described in WO2021031102.

### Pharmaceutical Compositions

In one embodiment, daprodustat or a pharmaceutically acceptable salt thereof is administered as a immediate release formulation such as an immediate release tablet.

In a more particular embodiment, an immediate release tablet of daprodustat or a pharmaceutically acceptable salt thereof is a formulation comprising from 1 to 8 mg (measured as the free acid) of daprodustat or a pharmaceutically acceptable salt thereof that meets the following dissolution criteria:
1. A mean (based on at least 12 tablets) of 85% or more of the of N-[(1,3-dicyclohexyl-6-hydroxy-2,4-dioxo-1,2,3,4-tetrahydro-5-pyrimidinyl)carbonyl]glycine free acid contained in the tablet dissolves within 45 minutes or less using United States Pharmacopeia (USP) Apparatus 2 with a rotational speed of 50±2 rpm and a dissolution volume of 500±5 mL for tablets containing <2 mg of N-[(1,3-dicyclohexyl-6-hydroxy-2,4-dioxo-1,2,3,4-tetrahydro-5-pyrimidinyl)carbonyl]glycine or a pharmaceutically acceptable salt thereof (measured as the free acid) and 900±9 mL for tablets containing ≥2 mg of N-[(1,3-dicyclohexyl-6-hydroxy-2,4-dioxo-1,2,3,4-tetrahydro-5-pyrimidinyl)carbonyl]glycine or a pharmaceutically acceptable salt thereof (measured as the free acid) in a pH 6.8 buffer or Simulated Intestinal Fluid USP without enzymes.

In one embodiment, the dissolution profile of an immediate release tablet comprising from 1 to 8 mg (measured as the free acid) of N-[(1,3-dicyclohexyl-6-hydroxy-2,4-dioxo-1,2,3,4-tetrahydro-5-pyrimidinyl)carbonyl]glycine or a pharmaceutically acceptable salt thereof or a pharmaceutically acceptable salt thereof using United States Pharmacopeia (USP) Apparatus 2 under the conditions specified above must additionally exhibit an f2 value≥50 compared to a tablet as described in Example 4 containing the same dose of active pharmaceutical ingredient. In one embodiment, the tablet of Example 4 was compacted using a main compaction pressure of 200-290 MPa, more particularly 240-260 MPa and even more particularly, about 250 MPa.

In a particular embodiment, the immediate release tablet comprises from 1 to 10 mg (measured as the free acid) daprodustat or a pharmaceutically acceptable salt thereof which has a tablet tensile strength of greater or equal to 1.7 MPa following compaction of the tablet core at a pressure in the range of 200 to 290 MPa. In more particular embodiments, the tablet tensile strength is greater than or equal to 1.75, 1.8, 1.9 or 2.0 MPa following compaction of the tablet core at a pressure in the range of 200 to 290 MPa. In particular embodiment, the immediate release tablet comprises from 1 to 8 mg (measured as the free acid) daprodustat or a pharmaceutically acceptable salt thereof.

In one embodiment, the immediate release tablet comprises a compartment containing daprodustat or a pharmaceutically acceptable salt thereof in an amount up to 5% based on the weight of the free acid, where the compartment does not contain a glidant. In one embodiment, the compartment contains the non solvated crystalline form of daprodustat free acid.

In one embodiment, the tablet is a monolithic tablet consisting of a single compartment of uniform composition that is optionally film coated. In one embodiment, the compartment is the tablet core. In another embodiment, the compartment is the entire tablet.

In an alternative embodiment, the tablet contains granules dispersed in an extragranular space and is optionally film coated. The granular and extragranular compositions may be different and form separate compartments. In one embodiment, the granular compartment is the compartment containing daprodustat or a pharmaceutically acceptable salt thereof (for example the non-solvated crystalline form of daprodustat free acid) and no glidant.

In one embodiment, the intragranular compartment comprises the crystalline form of non-solvated daprodustat free acid, a diluent, a binder and a disintegrant and no glidant. For the avoidance of doubt, more than one diluent, binder or disintegrant may be included. In one embodiment, the intragranular compartment consists of the crystalline form of non-solvated daprodustat free acid, one or more diluents, a binder and a disintegrant and no glidant.

In one embodiment, the extragranular compartment comprises a diluent, a disintegrant, a lubricant, and optionally a glidant. For the avoidance of doubt, more than one diluent, disintegrant, lubricant or glidant may be included. In one embodiment, the extragranular compartment consists of one or more diluents, a disintegrant, a lubricant, and optionally a glidant.

Suitable diluents include lactose, sucrose, dextrose, mannitol, sorbitol, starch (e.g. corn starch, potato starch, and pre-gelatinized starch), cellulose and its derivatives (e.g., microcrystalline cellulose), calcium sulfate, and dibasic calcium phosphate. In one embodiment, the diluent is not lactose.

Suitable binders include starch (e.g., corn starch, potato starch, and pre-gelatinized starch), hypromellose, gelatin, acacia, sodium alginate, alginic acid, tragacanth, guar gum, povidone, and cellulose and its derivatives (e.g. microcrystalline cellulose).

Suitable disintegrants include crospovidone, sodium starch glycolate, croscarmellose sodium, alginic acid, and sodium carboxymethyl cellulose.

Suitable lubricants include stearic acid, magnesium stearate, calcium stearate, and talc.

Glidants include colloidal silicon dioxide, talc, starch and magnesium stearate. In one embodiment, the glidant is colloidal silicon dioxide or magnesium stearate. In one embodiment, the glidant is silica. In another embodiment, the glidant is colloidal silicon dioxide.

In one embodiment, the invention provides an immediate release tablet, which tablet consists of:
a) intragranular components comprising the crystalline form of non-solvated daprodustat free acid, a diluent, a binder and a disintegrant; and
b) extragranular components comprising a diluent, a disintegrant, a lubricant, and optionally a glidant;
wherein the tablet is optionally coated.

In a more particular embodiment, the invention provides an immediate release tablet, which tablet consists of:
a) intragranular components consisting of the crystalline form of non-solvated daprodustat free acid and one or more diluents, one or more binders and one or more disintegrants; and
b) extragranular components comprising a diluent, a disintegrant, a lubricant, and optionally a glidant;
wherein the tablet is optionally coated.

A coating may be applied to the tablet core. An example of a commercially available coating is “OPADRY OY-S-28876 WHITE”. Coloured coatings are also commercially available.

In one embodiment, the immediate release tablet contains up to 76% by weight of intragranular components based on the weight of an uncoated tablet.

In one embodiment, the immediate release tablet comprises an intragranular compartment and an extragranular compartment wherein:
a. the intragranular components comprise:
   i. 1 to 10 mg of the crystalline form of non-solvated daprodustat free acid;
   ii. about 5 wt % hypromellose;
   iii. about 1.5 wt % croscarmellose sodium; and
   iv. mannitol and microcrystalline cellulose in a weight ratio from about 2.2 to about 3.6 (e.g., from about 2.3 to about 3.5, or about 2.25);
b. the extragranular components comprise, based on the total weight of the extragranular components:
   i. about 12 wt % croscarmellose sodium;
   ii. about 4 wt % magnesium stearate;
   iii. about 1.5% colloidal silica; and
   iv. mannitol and microcrystalline cellulose in a weight ratio from about 0.3 to about 3 (e.g. about 2).

In a particular embodiment, the tablet comprises about 1, 2 or 4 mg daprodustat and has a core tablet weight of about 150 mg. In another embodiment, the tablet comprises about 6 or 8 mg daprodustat and has a core tablet weight of about 300 mg. The tablets described herein may be optionally film-coated.

In one embodiment, the immediate release tablet does not comprise lactose.

### Medical Use

In one aspect, the invention provides daprodustat or a pharmaceutically acceptable salt thereof for use in reducing fatigue in a subject with anemia associated with chronic kidney disease which subject is not on dialysis.

In yet another embodiment, the invention provides use of daprodustat or a pharmaceutically acceptable salt thereof in the manufacture of the medicament for use in reducing fatigue in a subject with anemia associated with chronic kidney disease which subject is not on dialysis.

In another embodiment, the invention provides a method for reducing fatigue in a subject with anemia associated with chronic kidney disease, which method comprises administering to said subject daprodustat or a pharmaceutically acceptable salt thereof, wherein the subject is not on dialysis.

Suitably, the subject is a mammal. In a particular embodiment, the subject is human.

In more particular embodiments, the subject may be iron deficient (TSAT≤20% and/or serum ferritin≤100 ng/ml) and additionally receiving supplemental iron therapy.

In a further embodiment, the invention provides a dosage regimen for the treatment of fatigue in a subject with anemia associated with chronic kidney disease which aims to maintain haemoglobin in the range 10 to 12 g/dL and provide a gradual increase in haemoglobin levels where haemoglobin levels are below the target range. While the correlation between increases in either the vitality domain of SF-36 or the tired/low energy/weak domain of the CKD-AQ and haemoglobin increases is very weak, it is noted that a clinical study where the dose was adjusted based on haemoglobin concentration resulted in increases in both SF-36 Vitality Domain subscore and the score on the tired/low energy/weak domain of the CKD-AQ The dose is modified based on the concentration of haemoglobin determined at study visits. Haemoglobin concentration may be measured by known methods for example, a point of care analyser such as HemoCue or standard laboratory based testing.

In one aspect, the invention provides a dosage regimen for the treatment of fatigue in a subject with anemia associated with chronic kidney disease for patients wherein daprodustat or a pharmaceutically acceptable salt thereof is administered once daily at a dose of either 1 mg, 2 mg, 4 mg, 6 mg, 8 mg, 12 mg, 16 mg or 24 mg (based on daprodustat free acid) and wherein the dose is increased or decreased by one dose step based on the haemoglobin concentration of the subject to maintain the haemoglobin concentration of the subject within the range 10-12 g/dL. In one embodiment, the dose is increased or decreased by one dose step based on the haemoglobin concentration of the patient to maintain the haemoglobin concentration of the patient within the range 10-11 g/dL. In one embodiment, the dose is increased or decreased by one dose step based on the haemoglobin concentration of the patient to maintain the haemoglobin concentration of the patient at a target of 10 g/dL. In another embodiment, the dose is increased or decreased by one dose step based on the haemoglobin concentration of the patient to maintain the haemoglobin concentration of the patient within the range 11-12 g/dL.

In particular embodiments, the haemoglobin concentration of the subject is monitored at least once every three months. In more particular embodiments, the haemoglobin concentration of the subject is monitored monthly or every four weeks. The skilled person will appreciate that monitoring may be more frequent when treatment is initiated, with the frequency of monitoring decreasing once the haemoglobin concentration of the subject has stabilised within the target range/at the target (10 to 12 g/dL or 10 to 11 g/dL, 11 to 12 g/dL or 10 g/dL).

In embodiments when there is a rapid increase in the haemoglobin concentration of the patient (e.g. exceeding 2.0 g/dL within 4 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range and at risk of thromboembolism (e.g. where a patient has a history of prior stroke), the dose is reduced by one dose step or interrupted. In embodiments where the patient is exhibiting symptoms of anemia, the dose is increased by one dose step.

In embodiments where the patient is not on dialysis and the patient has previously been treated with an erythropoiesis stimulating agent (ESA), starting doses are based on prior ESA dosage. In embodiments where the patient is not on dialysis and the patient has not previously been treated with an erythropoiesis stimulating agent (ESA), starting doses are based on the patient's haemoglobin concentration. Table 1 sets out suitable starting doses.

TABLE 1

| Patients Switching from ESA Therapy | | | |
|---|---|---|---|
| Current ESA Dosage | | | |
| Epoetin | Darbepoetin | methoxy PEG-epoetin | |
| alfa IV | alfa SC/IV | beta SC/IV | Daprodustat |
| (units/week) | (mcg/4 week) | (mcg /month) | once daily |
| 1500 to 2000 | 20 to 30 | 30 to 40 | 1 mg |
| >2000 to <20,000 | >30 to 300 | >40 to 360 | 2 mg |
| ≥20,000 | >300 | >360 | 4 mg |

| | |
|---|---|
| Patients Not Receiving ESA Therapy | |
| Haemoglobin level (g/dL) | |
| <9 | 4 mg |
| ≥9 | 2 mg |

¹ESA: Erythropoiesis-stimulating agent

A dosage regimen for treatment of anemia due to chronic kidney disease to maintain haemoglobin concentration in the range 11-12 g/dL is provided, wherein the immediate release tablet of the invention is administered once daily at one of the following doses: 1, 2, 4, 6, 8, 12, 16 and 24 mg (dose of free acid), and wherein:
a) where the haemoglobin concentration≥13 g/dL, daprodustat therapy is ceased until the haemoglobin concentration<12 g/dL and therapy is commenced at one dose step lower;
b) where the haemoglobin concentration is in the range≥10.5 to <12.5 g/dL, the dose is maintained;
c) where the haemoglobin concentration is in the range>12 to ≤12.5 g/dL at two consecutive clinic visits and there has been an increase or no change in the haemoglobin concentration since the last visit, the dose is reduced by one dose step;
d) where the haemoglobin concentration is in the range>12.5 to <13 g/dL and there has been a decrease in haemoglobin concentration since the last visit, the dose is maintained;
e) where the haemoglobin concentration is in the range>12.5 to <13 g/dL and there has been an increase or no change in the haemoglobin concentration since the last visit, the dose is reduced by one dose step;
f) where the haemoglobin concentration is in the range≥10.5 to <11 at two consecutive clinic visits and there has been a decrease or no change in the haemoglobin concentration since the last visit, the dose is increased by one dose step;
g) where the haemoglobin concentration is in the range 8.5 to <10.5 g/dL and there has been an increase in haemoglobin concentration of ≥0.5 g/dL since the last visit, the dose is maintained;
h) where the haemoglobin concentration is in the range 8.5 to <10.5 g/dL and there has been a decrease, no change or an increase of <0.5 g/dL in haemoglobin concentration since the last visit, the dose is increased by one dose step;
i) where the haemoglobin concentration is <8.5 g/dL, the dose is increased by one dose step;
j) where there has been an increase in haemoglobin concentration of >2 g/dL over 4 weeks, or an increase in haemoglobin concentration of >1 g/dL over 2 weeks, the dose is reduced by one dose step; and
k) where there has been a decrease in haemoglobin concentration of >2 g/dL over 4 weeks, or a decrease in haemoglobin concentration of >1 g/dL over 2 weeks, the dose is increased by one dose step.

In one embodiment, daprodustat or a pharmaceutically acceptable salt thereof is administered once daily at one of the following doses: 1, 2, 4, 6, 8, 12, 16 and 24 mg (dose of free acid) in accordance with the dosage regimen described above.

For the avoidance of doubt, it is noted that any particular dose can be administered in a single tablet or multiple tablets. For example, the dose of 8 mg could be administered as a single 8 mg tablet, or two 4 mg tablets, or four 2 mg tablets, or eight 1 mg tablets, or a 6 mg and a 2 mg dosage form.

It will be apparent that dose adjustments will result in the daprodustat dose being increased or decreased by one dose step at a time. Those receiving the highest (maximum) dose of daprodustat who require a dose increase will maintain the same dose, while those receiving the lowest dose of daprodustat that require a dose decrease will discontinue daprodustat therapy.

### EXAMPLES

### Example 1

### Protocol

A 28 week randomized double blind placebo controlled clinical study in non-dialysis patient with anaemia associated with chronic kidney disease has been conducted. Participants were assessed for eligibility for inclusion in the study on the basis of the following (non-exhaustive) inclusion and exclusion criteria.

### Inclusion Criteria

≥18 years of age at the time of signing the informed consent
Stage 3, 4 or 5 Chronic Kidney Disease as assessed to the Kidney Disease Outcomes Quality Initiative (KDOQI)
Hemocue haemoglobin from 8.5-10.5 g/dL at screening (4 weeks prior to randomization) and from 8.5-10.0 g/dL on date of randomization (day 1)
Up to one IV iron dose within 8 weeks prior to screening and NO IV iron use between screening and randomization
If needed, stable maintenance oral iron supplementation is permitted. There should be <50% change in overall dose and no change in type of iron in the 4 weeks prior to Day 1 randomization visit.
Female participants of childbearing potential, provided that they are not pregnant or breastfeeding and provided they agree to follow contraceptive guidance during the treatment period and for 4 weeks after the last dose of study treatment
Capable of giving informed consent

### Exclusion Criteria

On dialysis or clinical evidence of impending need to initiate dialysis within 180 days after randomization
Planned kidney transplant within 28 days after randomization
Transferrin saturation<15% at screening
Ferritin<50 ng/ml at screening
Recombinant erythropoietin use within the 8 weeks prior to screening or between screening and randomization

Enrolled participants were randomized 1:1 to receive either daprodustat or matching placebo tablets administered once daily. The starting dose or matching placebo were assigned based on the Hemocue haemoglobin concentration at randomization such that participants having a haemoglobin concentration of 8.5 to <9 g/dL had a daily starting dose of 4 mg, and participants having a haemoglobin concentration of 9 to 10 g/dL had a daily starting dose of 2 mg.

The treatment doses for participants in both treatment arms were titrated based on haemoglobin concentration as assessed by HemoCue. Haemoglobin concentration by HemoCue is assessed on day 1, and at weeks 2, 4, 8, 12, 16, 20, 24 and 28. The available dose steps of daprodustat and placebo were 1 mg, 2 mg, 4 mg, 6, mg, 8 mg, 10 mg, 12 mg and 16 mg. Dose modifications for participants followed the dose adjustment algorithm to achieve and maintain haemoglobin concentration within the target range of 11.0 to 12.0 g/dL outlined in Table 2.

### Table 2

TABLE 2

| HemoCue haemoglobin | HemoCue haemoglobin | |
|---|---|---|
| concentration (g/dL) at current | concentration change since last | |
| study visit | study visit | Dose adjustment |
| <8.5 | Any change | Increase dose to next higher |
| | | dose step and evaluate for |
| | | rescue criteria |
| 8.5 to <10.5 | Increase of <0.5 g/dL, or any | Increase dose to next higher |
| | decrease, or no change | dose step |
| 8.5 to <10.5 | Increase of ≥0.5 and ≤2 g/dL | Maintain dose |
| ≥10.5 to ≤12.5 | Any change ≤2 g/dL | Maintain dose |
| ≥10.5 to ≤11 at two | Decreasing or no change | Increase to the next higher |
| consecutive visits | | dose step |
| ≥12.0 to ≤12.5 at two | Increasing or no change | Decrease to the next lower |
| consecutive visits | | dose step |
| >12.5 to <13 | Increasing or no change | Decrease to the next lower |
| | | dose step |
| ≥13.0 | Any change | Repeat haemoglobin |
| | | concentration and average |
| | | values. If confirmed, |
| | | temporarily hold the dose and |
| | | re-check haemoglobin at the |
| | | next study visit. Restart at one |
| | | dose steo lower when |
| | | Haemoglobin <12.0 g/dL |
| | | provided iit has been at least 2 |
| | | weeks from the prior study visit |
| Any | >2 g/dL increase over 4 weeks | Repeat haemoglobin |
| | or >1 g/dL increase over 2 | concentration and average |
| | weeks | values. If confirmed, decrease |
| | | to the next lower dose step |
| Any | >2 g/dL decrease over 4 weeks | Repeat haemoglobin |
| | or >1 g/dL decrease over 2 | concentration and average |
| | weeks | values. If confirmed, increase |
| | | to the next higher dose step |

Iron status of the participants was assessed at day 1 and on weeks 16 and 28. Iron therapy was administered starting with oral iron if ferritin<50 ng/ml and/or transferrin saturation is <15% to re establish participants screening iron parameters. IV iron could be administered to participants who were intolerant to oral iron, otherwise IV iron was only administered to participants being evaluated for rescue.

A rescue algorithm was provided to minimize participants having an inadequate response to treatment of their anemia for an extended period of time. Starting from week 4, where haemoglobin concentration as assessed by HemoCue was either <7.5 g/dL, or <8.5 g/dL where the participant is symptomatic, or <8.5 g/dL on three consecutive visits, then the participant was evaluated for iron status and rescue. Initial intervention was dependent upon iron status such that if the participant had a TSAT<15% and/or ferritin<50 ng/ml from a prior study visit then a single dose of IV iron of up to 1000 mg was administered in addition to iron administered following the iron management criteria. Where the patient was iron replete (TSAT≥15% and ferritin≥50 ng/ml) or where haemoglobin concentration remains <8.5 g/dL at the next study visit despite IV iron use, study treatment should be discontinued and the participant should be rescued according to local clinical practice.

The primary objective of the trial was to compare the efficacy of daprodustat to placebo on mean change in haemoglobin concentration. The primary endpoint was the mean change in haemoglobin concentration between baseline (day 1) and the evaluation period (mean over week 24 to week 28 inclusive).

Several secondary and exploratory objectives were explored. Secondary objectives included comparison of daprodustat to placebo on health related quality of life and comparison of daprodustat to placebo for improving symptoms of anemia of chronic kidney disease.

The Short form SF-36 questionnaire was completed by participants on day 1 and on weeks 8, 12 and 28, and the CKD-AQ questionnaire was completed by participants on week −2 (i.e. during screening period) and on day 1 and weeks 8, 12 and 28.

### Endpoints Included:

Mean change in the SF-36 Vitality Domain score (0-100) between baseline and week 28. This was a principal secondary endpoint and the study was appropriately powered for this endpoint.
Mean change from baseline by domain and overall symptoms score on the CKD-AQ (the study was not powered for this secondary endpoint, so only nominal significance can be assessed).

In addition to analysis of the intention to treat population, several sub-group analyses were pre-specified including subgroups with baseline hsCRP quartiles of <0.9 mg/L, 0.9 to <2.6 mg/L, 2.60 to <6.60 mg/L and ≥6.60 mg/L.

For the endpoints of mean change in haemoglobin and SF-36 Vitality Domain, an analysis of covariance model was used including covariates of baseline level, treatment, and region. The model provided point estimate and two-sided 95% CI for the treatment effect, together with a one-sided p-value. Significance was determined if the one-sided p-value was less than 0.025. The analysis population for the haemoglobin and SF-36 Vitality Domain endpoints was the intent-to-treat population. The analysis population for the Chronic Kidney Disease Anemia Questionnaire Tired/Low Energy/Weak Domain endpoint was the on treatment population and analysis was based on a mixed model repeated measures model fitted from baseline up to Week 28 with factors for treatment, time, region, baseline value, baseline value by time, and treatment by time interactions.

For the endpoints of mean change in haemoglobin and SF-36 Vitality Domain by subgroup, a similar model to that described above was used, adding covariates for subgroup and subgroup by treatment interaction term. The analysis population for these endpoints was the intent-to-treat population.

For endpoints of difference in SF-36 Vitality domain response rate, a Cochran-Mantel-Haenszel chi-squared test was used adjusting for treatment, baseline level, and region, to make comparisons between the treatment groups. Significance was determined if the one-sided p-value was less than 0.025. The analysis population for these endpoints was the intent-to-treat population.

Analyses were calculated using SAS software, Version 9.2 (or higher).

### Results

Daprodustat significantly increased SF-36 Vitality Sub-score compared to placebo in this study. As shown in Table 3, the adjusted mean SF-36 Vitality Change over the treatment period was 7.29 for daprodustat in the intention to treat population (a within subject change of 6 points is conservatively estimated to be clinically relevant, see Example 3). The adjusted mean treatment difference (dapro-placebo) was 5.36. The one sided superiority p-value (based on test of null hypothesis: (Dapro—Placebo)<=0 vs alternative: difference>0) was 0.0005. 58% of participants treated with daprodustat demonstrated a ≥6.0 point difference from baseline to week 28 in the SF-36 Vitality Sub-score, compared to 40% for participants treated with placebo. The adjusted difference in response rate (dapro-placebo) was 0.13 (0.04, 0.22), which was nominally significant (one sided p value=0.0049). It is noted for completeness that missing or off-treatment SF36 data was imputed.

TABLE 3

| | Placebo | Daprodustat |
|---|---|---|
| Number of subjects with baseline and | 307 | 307 |
| week 28 SF-36 Vitality Sub-score | | |
| (includes imputed values) | | |
| Baseline SF-36 Vitality Sub-score Mean | 52.23 (21.070) | 50.74 (21.175) |
| (SD) | | |
| Week 28 SF-36 Vitality Sub-score | 53.78 (17.011) | 58.40 (18.312) |
| Mean (SD) | | |
| Adjusted mean SF-36 Vitality Change | 1.93 (1.161) | 7.29 (1.121) |
| from Baseline at Week 28 (SE) | | |
| Adjusted Mean Treatment Difference | 5.36 | |
| (dapro − placebo) | | |
| Two sided 95% CI for adjusted mean | 2.17, 8.56 | |
| difference | | |
| One sided p-value (based on test of null | 0.0005 | |
| hypothesis: (Dapro − Placebo) <=0 vs | | |
| alternative: difference >0) | | |

Daprodustat increased SF-36 Vitality Sub-score compared to placebo regardless of baseline hsCRP level. Subgroups with baseline hsCRP<0.9 mg/L, 0.9 to <2.6 mg/L, 2.60 to <6.60 mg/L and ≥6.60 mg/L exhibited an adjusted mean SF-36 Vitality Change of 9.84, 4.98, 7.92 and 8.24 and an adjusted mean treatment difference (CI) of 10.0 (3.53, 16.47), 1.09 (−4.86, 7.04), 4.38 (−1.77, 10.53) and 7.71 (1.62, 13.80) respectively.

Daprodustat also increased the score on the Tired/Low Energy/Weak domain of the Chronic Kidney Disease—Anemia Questionnaire. As shown in Table 4, the adjusted mean change over the treatment period was 8.72 for daprodustat. The adjusted mean treatment difference (dapro-placebo) was 5.91. The one sided p-value (based on test of null hypothesis: (Dapro—Placebo)<=0 vs alternative: difference>0) was <0.0001.

TABLE 4

| | Placebo | Daprodustat |
|---|---|---|
| Number of subjects with baseline and week | 193 | 212 |
| 28 score on the CKD-AQ Tired/Low | | |
| Energy/Weak Domain | | |
| Adjusted mean change from baseline to week | 2.81 (1.132) | 8.72 (1.086) |
| 28 on CKD-AQ Tired/Low Energy/Weak | | |
| Domain (SE) | | |
| Adjusted Mean Treatment Difference | 5.91 | |
| (dapro − placebo) | | |
| Two sided 95% CI for adjusted mean | 2.83, 9.00 | |
| difference | | |
| One sided p-value (based on test of null | <0.0001 | |
| hypothesis: (Dapro − Placebo) <=0 vs | | |
| alternative: difference >0) | | |

These results demonstrate that daprodustat is responsible for improvements in patient reported measures of fatigue over the treatment period, as measured by two separate instruments, the SF-36 Vitality Domain (statistically significant) and the Tired/Low Energy/Weak domain of the CKD-AQ (nominally significant in view of the fact that the study was not powered for this endpoint).

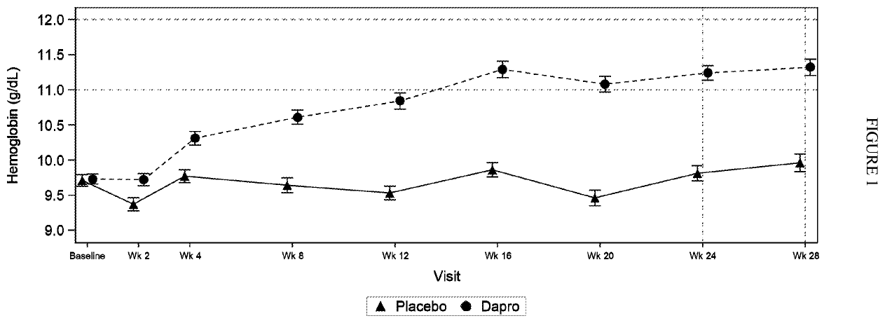

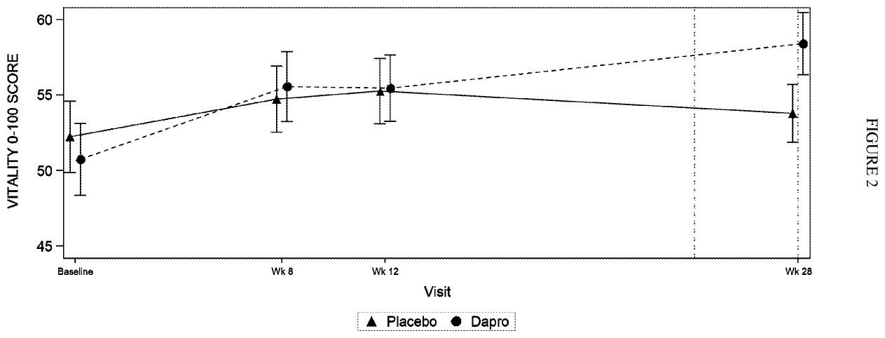

Daprodustat additionally significantly increased haemoglobin concentration compared to placebo over the treatment period (the one sided superiority p-value (based on test of null hypothesis: (Dapro—Placebo)<=0 vs alternative: difference>0)<0.0001). However, the time course of increase in haemoglobin concentration did not closely mirror the time course of improvement of fatigue as measured by the SF-36 Vitality Domain. FIGS. 1 and 2 show the changes in haemoglobin concentration (FIG. 1) and SF-36 vitality domain scores (FIG. 2) in daprodustat and placebo treated patients. At the 12 week time point, there was a clear difference in haemoglobin response between daprodustat and placebo (see FIG. 1). However, at week 12, the SF-36 vitality domain scores for daprodustat and placebo were not distinguishable, with significant treatment differences in SF-36 vitality domain scores only achieved at week 28 (see FIG. 2). A post-hoc analysis (Example 3) was subsequently used to explore the correlation between on treatment change from baseline to week 28 haemoglobin concentration (g/dL) vs. on treatment change from baseline to week 28 for the patient reported measures of fatigue (SF36 Vitality Domain Sub-score and the Tired/Low Energy/Weak domain of the Chronic Kidney Disease—Anemia Questionnaire). The results are presented in Example 3.

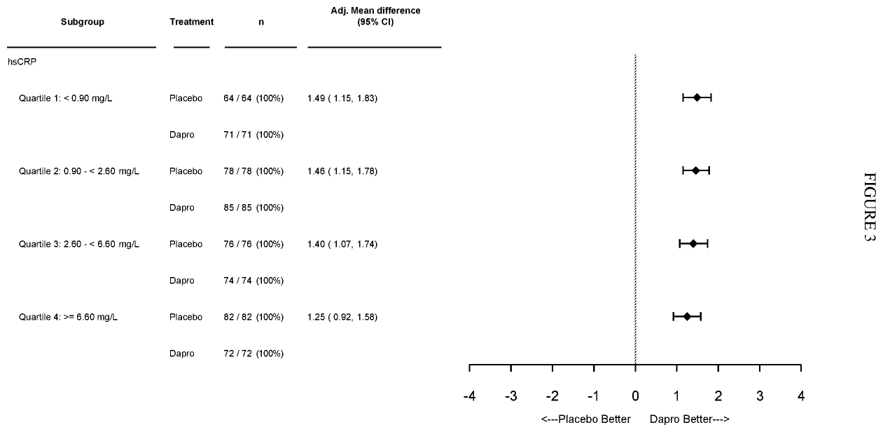

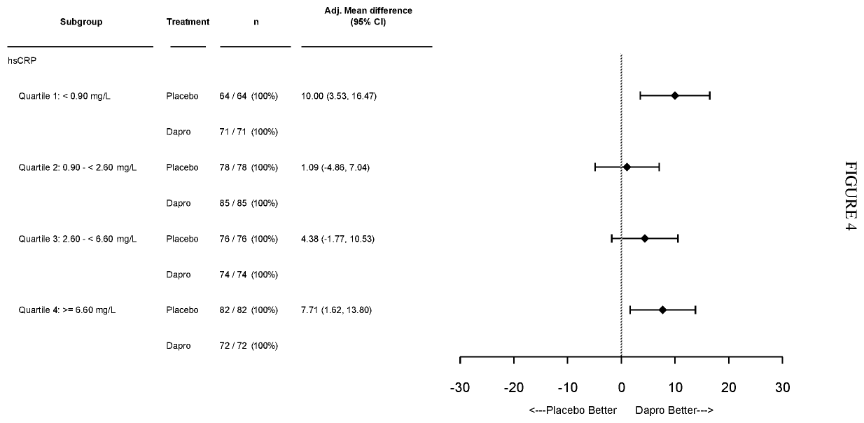

Patient populations were identified in which reductions in fatigue did not closely correlate with increases in haemoglobin concentration. FIG. 3 shows a Forest Plot of on treatment adjusted mean SF-36 Vitality Change for each hsCRP quartile. The corresponding Forest Plot showing on treatment adjusted mean haemoglobin change for each hsCRP quartile is given in FIG. 4. This shows that the patient population having hsCRP≥6.60 mg/L exhibits a large change in SF-36 vitality domain subscore (adjusted mean treatment difference=7.71, 95% confidence intervals 1.62, 13.80) that would be expected to be clinically relevant (see Example 3) with a relatively modest haemoglobin increase (adjusted mean treatment difference=1.25, 95% confidence intervals 0.92, 1.58). In this patient population, the observed, clinically relevant reductions in fatigue (improvements in vitality) are not in large part correlated with haemoglobin increase.

The changes in hsCRP levels in each quartile are given below in Tables 5 to 8.

TABLE 5
hsCRP <0.9 mg/L

| | | | Geometric mean | |
|---|---|---|---|---|
| Treatment | Timepoint | n | hsCRP (mg/L) | Geo-CV % |
| Placebo | Day 1 | 64 | 0.47 | 42.672 |
| | Baseline | 64 | 0.47 | 42.672 |
| | Week 28 | 42 | 1.43 | 266.308 |
| | End of Treatment | 42 | 1.43 | 266.308 |
| Daprodustat | Day 1 | 71 | 0.48 | 44.753 |
| | Baseline | 71 | 0.48 | 44.753 |
| | Week 28 | 48 | 0.85 | 91.172 |
| | End of Treatment | 49 | 0.86 | 89.911 |

TABLE 6
hsCRP 0.9-<2.60 mg/L

| | | | Geometric mean | |
|---|---|---|---|---|
| Treatment | Timepoint | n | hsCRP (mg/L) | Geo-CV % |
| Placebo | Day 1 | 78 | 1.45 | 27.801 |
| | Baseline | 78 | 1.45 | 27.801 |
| | Week 28 | 46 | 1.90 | 230.304 |
| | End of Treatment | 46 | 1.90 | 230.304 |
| Daprodustat | Day 1 | 85 | 1.46 | 34.270 |
| | Baseline | 85 | 1.46 | 34.270 |
| | Week 28 | 62 | 2.55 | 148.895 |
| | End of Treatment | 62 | 2.55 | 148.895 |

TABLE 7
hsCRP 2.60-<6.60 mg/L

| | | | Geometric mean | |
|---|---|---|---|---|
| Treatment | Timepoint | n | hsCRP (mg/L) | Geo-CV % |
| Placebo | Day 1 | 76 | 3.97 | 26.871 |
| | Baseline | 76 | 3.97 | 26.871 |
| | Week 28 | 45 | 4.54 | 159.156 |
| | End of Treatment | 47 | 4.56 | 153.205 |
| Daprodustat | Day 1 | 74 | 3.93 | 25.873 |
| | Baseline | 74 | 3.93 | 35.873 |
| | Week 28 | 54 | 3.85 | 106.394 |
| | End of Treatment | 55 | 3.83 | 105.175 |

TABLE 8
hsCRP ≥6.60 mg/L

| | | | Geometric mean | |
|---|---|---|---|---|
| Treatment | Timepoint | n | hsCRP (mg/L) | Geo-CV % |
| Placebo | Day 1 | 82 | 15.05 | 80.058 |
| | Baseline | 82 | 15.05 | 80.058 |
| | Week 28 | 49 | 6.18 | 195.488 |
| | End of Treatment | 52 | 6.20 | 192.127 |
| Daprodustat | Day 1 | 72 | 16.79 | 90.518 |
| | Baseline | 72 | 16.96 | 91.848 |
| | Week 28 | 51 | 9.04 | 150.431 |
| | End of Treatment | 52 | 8.92 | 148.934 |

Where baseline levels of hsCRP are <6.60 mg/L, changes in hsCRP levels are modest (see Tables 5-7). In subjects with high baseline hsCRP (≥6.60 mg/L), there were large reductions in hsCRP levels in the daprodustat group raising the possibility that increases in vitality may be linked with hsCRP reduction in this group (Table 8). However, no firm conclusions can be drawn due to the fact that similar reductions in hsCRP levels were also observed in the placebo group with high baseline hsCRP (Table 8). It is also possible that the reductions observed may (at least in part) be due to the subjects having the highest CRP levels not being evaluated at week 28/end of treatment, possibly because these subjects were rescued.

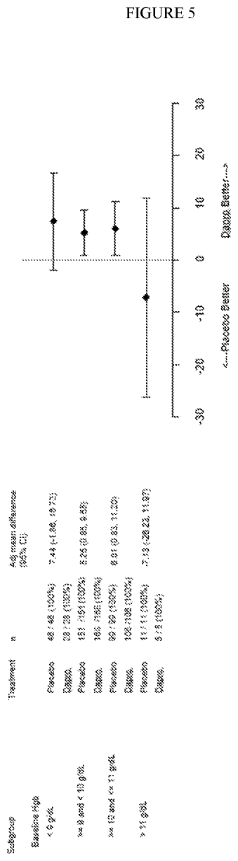

In addition, baseline haemoglobin concentration has an impact upon adjusted mean treatment in SF-36 Vitality Sub-score. FIG. 5 shows a Forest Plot of on treatment adjusted mean SF-36 Vitality Change for subjects having baseline haemoglobin concentration of <9 g/dL, ≥9 to <10 g/dL, ≥10 to ≤11 g/dL and >11 g/dL. Whilst the small number of subjects in the >11 g/dL complicates interpretation, it is noted that there was a decrease in adjusted mean treatment difference in the SF36 Vitality in this subgroup (adjusted mean treatment difference −7.13, confidence intervals −26.23, 11.97). A smaller increase in haemoglobin concentration, resulting from study design was also observed in this subgroup (adjusted mean treatment difference 0.76 g/dL, confidence intervals −0.36, 1.88). Whilst study design and the small number of subjects having haemoglobin>11 g/dL at baseline prevents firm conclusions being reached, it seems likely that increases in SF36 vitality scores are most pronounced in subjects having baseline haemoglobin concentration of to ≤11 g/dL.

### Example 2

Post-hoc analyses were carried out on data from the study described in Example 1 to determine the correlation of on treatment change from baseline to week 28 in haemoglobin concentration (g/dL) vs. on treatment change from baseline to week 28 in SF-36 vitality Sub-score and the correlation of on treatment change from baseline to week 28 in haemoglobin concentration (g/dL) vs. on treatment change from baseline to week 28 in Tired/Low Energy/Weak domain of the CKD-AQ. Correlations were calculated using SAS software, Version 9.2 (or higher). Statistical output included Pearson correlation coefficient, correlation estimate based on Pearson Correlation Statistics (Fisher's z Transformation) with bias adjustment, two-sided 95% CI, and a one-sided p-value. The one-sided p-value was based on the test of the null hypothesis: Rho<=0.40 vs. the alternative hypothesis: Rho>0.40. Nominal significance would be established if the one-sided p-value was less than 0.025.

The number of subjects having both baseline and week 28 haemoglobin and SF-36 vitality sub-scores was 188 (placebo) and 210 (dapro). The calculated Pearson correlation co-efficient for on treatment change from baseline to week 28 in haemoglobin concentration (g/dL) vs. on treatment change from baseline to week 28 in SF-36 vitality Sub-score was 0.0356, and the correlation estimate (based on Pearson Correlation Statistics (Fisher's z transformation with bias adjustment was 0.0356 (two sided 95% confidence intervals: −0.06, 0.13). The one sided p value was >0.999.

Similarly, the number of subjects having both baseline and week 28 haemoglobin and CKD—AQ Tired/Low Energy/Weak Domain scores was 191 (placebo) and 212 (dapro). The calculated Pearson correlation co-efficient for on treatment change from baseline to week 28 in haemoglobin concentration (g/dL) vs. on treatment change from baseline to week 28 in Tired/Low Energy/Weak domain of the CKD-AQ was 0.0877, and the correlation estimate (based on Pearson Correlation Statistics (Fisher's z transformation with bias adjustment was 0.0876 (two sided 95% confidence intervals: −0.01, 0.18). The one sided p value was >0.999.

These post-hoc analyses confirm a very weak correlation between changes in haemoglobin concentration and fatigue as measured by either the SF-36 Vitality Sub-score or the Tired/Low Energy/Weak Domain of the CKD-AQ.

### Example 3

A targeted literature review was conducted to identify thresholds for meaningful change (minimal clinically important difference or MCID) for the SF-36 vitality scale (k=4) and/or its four constituent items. Searches were conducted in MEDLINE (via PubMed) and Embase for articles and conference abstracts published in English from Jan. 1, 1996 through and including Jan. 26, 2018. A total of 56 citations were identified using search terms intended to capture literature directed to minimal clinically important difference of the SF-36 vitality scale, and 33 were identified using the search terms intended to capture literature directed to minimal clinically important difference of the KDQOL (Kidney Disease Quality Of Life instrument) since the SF-36 is embedded within the KDQOL. A search of the grey literature was also conducted to attempt to capture information from sources not indexed in MEDLINE or Embase that was otherwise available from various scientific conferences or websites. The grey literature search involved using common search engines (Google and Mozilla) to identify any MCIDs for the SF-36 vitality scale not identified in MEDLINE or Embase. In addition, the grey literature search involved searches of conference abstracts and poster presentations (as available) from the 2015-2017 meetings of the below conferences and congresses.
World Nephrology Congress
American Society of Nephrology (ASN) Annual Meeting (“Kidney Week”)
European Renal Association—European Dialysis and Transplant Association (ERA-EDTA) Congress
National Kidney Foundation Clinical Meeting
International Society for Pharmacoeconomics and Outcomes Research
International Society for Quality of Life Research

In addition to meeting the study selection criteria above, the additional requirement for grey literature sources to be included in the review was that they are citable in an acceptable format for regulatory agencies. The search of the grey literature yielded no additional unique citations.

The following criteria were employed to identify hits that justified greater review.
Relates to adult patients with chronic kidney disease
Uses a licensed treatment
Includes as an outcome an empirical estimate of minimal clinically important difference for the SF-36 vitality subscale and/or its four constituent items
Relates to a randomized controlled trial, an observational study, or a targeted or systematic literature review
In the English language (any geographical region)

Screening of the hits using the criteria was conducted by the reviewers using the following process.
Level 1: Review of title/abstract screening by one investigator
Level 2: Review of full text. Any rejected articles were reviewed by an independent investigator and any discrepancies were resolved.
Level 3: Abstraction of the following data:
   Author and year
   Country
   Study design
   Disease(s) studied
   Sample size
   Inclusion criteria
   Exclusion criteria
   Duration of follow-up (mean, median or maximum)
   Treatment studied
   Patient demographic characteristics
   Specific MCID methodology
   Scoring of the SF-36 (norm-based vs. traditional 0-100 based)
   MCID results for the SF-36 vitality scale and/or its four constituent items

Following data extraction and tabulation, the MCID results for the SF-36 vitality scale and/or its four constituent items were graphically displayed and tabulated to permit triangulation across MCID methods.

For the SF-36 citations: (1) 11 conference abstracts were screened and one was subject to full extraction; and (2) 45 regular articles were screened, 10 were subject to full-text review, and three were abstracted. For the KDQOL citations: (1) eight conference abstracts were screened and zero were subject to full extraction; and (2) 25 regular articles were screened, 15 were subject to full-text review, and two were abstracted. The reference lists from four systematic reviews/meta-analyses were reviewed to identify other relevant articles related to a MCID for the SF-36 vitality scale in CKD. The reference list of every abstracted article was also searched. A total of 16 additional studies were identified through these backward citation searches.

Four principal methodological categories were derived for describing the articles that reported thresholds for meaningful change for the SF-36 vitality scale in CKD:
Within-group treatment studies-longitudinal studies that involved one or more treatments for CKD anemia where within-group change from baseline was reported for the SF-36 vitality scale
Studies investigating target hemoglobin level (correction or normalization of hemoglobin) and within-group change in the SF-36 vitality scale
Psychometric studies addressing distribution-based methods of deriving a MCID
Psychometric studies addressing anchor-based methods of deriving a MCID

Considering the inclusion of distribution-based results, the overall threshold for meaningful change for the SF-36 vitality scale in CKD derived from the 21 abstracted studies is an unweighted mean of 5.8, a weighted mean of 5.3, an unweighted median of 5.6, and a weighted median of 4.5. Excluding distribution-based results, the overall threshold for meaningful change for the SF-36 vitality scale in CKD derived from 16 abstracted studies is an unweighted mean of 6.1, a weighted mean of 5.7, unweighted median of 5.8, and a weighted median of 4.9.

Based on this study, a literature-based threshold for meaningful change for the SF-36 vitality scale applied to psychometric analyses of 6.0 is deemed appropriate.

### Example 4

Tablet formulations of daprodustat free acid may be prepared as follows. The tablet cores comprise granules and extragranular components. Granules are prepared by adding daprodustat, mannitol, microcrystalline cellulose, hypromellose 2910 and croscarmellose sodium into a high shear granulator. The powders are blended under high shear for at least 5 minutes and granulation performed while spraying at least 26% w/w purified water over a water addition time of at least 7 minutes and wet massing time of at least 2 minutes. The wet granules are dried in a fluid bed dryer to a target moisture content of not exceeding 2% w/w at a product temperature of at least 38° C. and the granules are dry milled to normalize granule size distribution. The milled granules are further blended with extragranular components mannitol, microcrystalline cellulose, croscarmellose sodium and glidant colloidal silicon dioxide. Magnesium stearate is added and the resulting mixture is compressed using compaction pressures in the range 180 to 370 MPa into tablet cores using a rotary tablet press under the following conditions.

Tablet shape/size: round, biconvex tablets/7 mm diameter (≤4 mg); 9 mm diameter (≥6 mg) Compression speed of at least 40000 tablets per hour

The compositions of the tablets are provided in Table 9.

TABLE 9

| | Quantity (mg/tablet) | | | | |
|---|---|---|---|---|---|
| Component | 1 mg | 2 mg | 4 mg | 6 mg | 8 mg |

Granules

| | | | | | |
|---|---|---|---|---|---|
| Daprodustat | 1.00 | 2.00 | 4.00 | 6.00 | 8.00 |
| Mannitol | 72.30 | 71.60 | 70.22 | 141.83 | 140.45 |
| Microcrystalline Cellulose | 31.88 | 31.58 | 30.96 | 62.54 | 61.92 |
| Hypromellose 2910 | 5.63 | 5.63 | 5.63 | 11.25 | 11.25 |
| Croscarmellose Sodium | 1.69 | 1.69 | 1.69 | 3.38 | 3.38 |
| Purified Water | — | — | — | — | — |

Extragranular Components

| Mannitol | 20.44 | 20.44 | 20.44 | 40.87 | 40.87 |
|---|---|---|---|---|---|
| Microcrystalline Cellulose | 10.50 | 10.50 | 10.50 | 21.00 | 21.00 |
| Croscarmellose Sodium | 4.50 | 4.50 | 4.50 | 9.00 | 9.00 |
| Colloidal Silicon Dioxide | 0.56 | 0.56 | 0.56 | 1.13 | 1.13 |
| Magnesium Stearate | 1.50 | 1.50 | 1.50 | 3.00 | 3.00 |
| Core tablet weight | 150.0 | | | 300.0 | |
| (mg) | | | | | |

Purified water for granulation is removed during processing and does not remain in the tablet.

### Example 5

Questions in the Tired/Low energy/Weak Domain of the CKD-AQ are given in Table 10:

TABLE 10

| | |
|---|---|
| Item wording | Response options |

GENERAL INSTRUCTIONS
People with chronic kidney disease sometimes experience anemia, resulting from a decrease
in red blood cells.
The following questions ask you to think about how you have been feeling, and how easy
or difficult it has been for you to do your daily activities.
There are no right or wrong answers. Please answer all questions and select the one
response that best matches your experience.
The following questions ask about how much of the time you may have experienced each
symptom in the past 7 days.

| | |
|---|---|
| 1. In the past 7 days, how much of the | a) None of the time |
| time were you very tired? | b) A little bit of the time |
| | c) Some of the time |
| | d) Most of the time |
| | e) All of the time |
| 2. In the past 7 days, how much of the | a) None of the time |
| time were you low or no energy? | b) A little bit of the time |
| | c) Some of the time |
| | d) Most of the time |
| | e) All of the time |
| 3. In the past 7 days, how much of the | a) None of the time |
| time did you feel weak? | b) A little bit of the time |
| | c) Some of the time |
| | d) Most of the time |
| | e) All of the time |

In the last section, you answered questions about how FREQUENTLY you had each symptom. For
the following questions, please indicate how SEVERE each of the following symptoms were in the
past 7 days.

| | |
|---|---|
| 9. Rate the severity of feeling very tired in | 0 1 2 3 4 5 6 7 8 9 10 |
| the past 7 days | Absent/I did not have Worst imaginable |
| 10. Rate the severity of feeling low or no | 0 1 2 3 4 5 6 7 8 9 10 |
| energy in the past 7 days | Absent/I did not have Worst imaginable |
| 11. Rate the severity of feeling weak in | 0 1 2 3 4 5 6 7 8 9 10 |
| the past 7 days | Absent/I did not have Worst imaginable |

The following questions ask you to think about how you have been feeling and how easy or difficult
it has been for you to do your daily activities. Please answer these questions thinking about the
past 7 days.

| | |
|---|---|
| 19. In the past 7 days, how much of the | a) None of the time |
| time did you feel you didn't want to do | b) A little bit of the time |
| anything because you were tired? | c) Some of the time |
| | d) Most of the time |
| | e) All of the time |
| 20. In the past 7 days, how much of the | a) None of the time |
| time did you need to take a break or stop | b) A little bit of the time |
| doing an activity because you were | c) Some of the time |
| tired? | d) Most of the time |
| | e) All of the time |
| 21. In the past 7 days, how much of the | a) None of the time |
| time did you need to lie down or take a | b) A little bit of the time |
| nap because you were tired? | c) Some of the time |
| | d) Most of the time |
| | e) All of the time |
| 22. In the past 7 days, how much of the | a) None of the time |
| time were you distressed about being | b) A little bit of the time |
| tired? | c) Some of the time |
| | d) Most of the time |
| | e) All of the time |

As can be seen, frequency items have a five-level frequency categorical rating scale of “none of the time,” “a little bit of the time,” “some of the time,” “most of the time,” and “all of the time”. Severity items have an 11-point numerical rating scale (0 to 10) with the extreme endpoints labeled with verbal descriptors: 0=“absent/did not have” to 10=“worst imaginable.”

Because of the distinct rating scales, it is necessary to recalibrate the frequency and severity items to a 0-100 scale so “methods factors” did not confound analyses. A simple linear transformation was used to recalibrate the items to a 0-100 scale as outlined in Tables 11 and 12.

TABLE 11

| | CKD-AQ Frequency Items | | | | |
|---|---|---|---|---|---|
| Original Scoring | 0 | 1 | 2 | 3 | 4 |
| Linear Transformation | 100 | 75 | 50 | 25 | 0 |

TABLE 12

| | CKD-AQ Severity Items | | | | | | | | | | |
|---|---|---|---|---|---|---|---|---|---|---|---|
| Original Scoring | 0 | 1 | 2 | 3 | 4 | 5 | 6 | 7 | 8 | 9 | 10 |
| Linear | 100 | 90 | 80 | 70 | 60 | 50 | 40 | 30 | 20 | 10 | 0 |
| Transformation | | | | | | | | | | | |

To obtain the domain score the linear transformation scores are summed and then divided by the number of items in the domain. The domain score is always between 0 and 100.

1-7. (canceled)

8. A method for reducing fatigue in a human subject with anemia associated with chronic kidney disease, which method comprises administering to said subject, daprodustat or a pharmaceutically acceptable salt thereof, wherein the human subject is not on dialysis.

9. The method according to claim 8, wherein the human subject with anemia has a haemoglobin concentration of ≤11 g/dL at baseline.

10. The method according to claim 9, wherein the human subject has hsCRP≥6.60 mg/L at baseline.

11. The method according to claim 10, wherein fatigue is reduced when the mean score on the vitality subscale of the SF-36 questionnaire is increased at the end of the treatment period relative to baseline in a population of subjects.

12. The method according to claim 10, wherein fatigue is reduced when the mean increase on the vitality subscale of the SF-36 questionnaire is at least 6 points at the end of the treatment period relative to baseline in a population of subjects.

13. The method according to claim 10, wherein fatigue is reduced when the percentage of subjects achieving an increase of at least 6 points on the vitality subscale of the SF-36 questionnaire is greater than 50%.

14. A method according to claim 10, wherein fatigue is reduced where the mean score in the tired/low energy/weak domain of the CKD-anaemia questionnaire is increased at the end of the treatment period relative to baseline in a population of subjects.
